(19) United States
(12) Patent Application Publication
(10) Pub. No.: US 2008/0139870 A1
(43) Pub. Date: Jun. 12, 2008

(54) Systems and methods for treating patient hypertonicity

(75) Inventors: Bradford E. Gliner, Sammamish, WA (US); Brad Fowler, Duvall, WA (US); Justin Hulvershorn, Seattle, WA (US); Leif R. Sloan, Seattle, WA (US)

Correspondence Address: PERKINS COIE LLP;PATENT-SEA, P.O. BOX 1247, SEATTLE, WA, 98111-1247, US

(73) Assignee: Northstar Neuroscience, Inc., Seattle, WA (US)

(21) Appl. No.: 11/638,326

(22) Filed: Dec. 12, 2006

(51) Int. Cl. A61N 2/02

(52) U.S. Cl. 600/12

(57) ABSTRACT

Systems and methods for treating patient hypertonicity are disclosed. A method in accordance with one embodiment includes identifying a cortical target neural population associated with hypertonicity in a patient, and reducing or eliminating patient hypertonicity by applying electromagnetic signals to the target neural population. In further particular embodiments, the electromagnetic signals are first electromagnetic signals, and the method can further include reducing or eliminating an additional patient dysfunction by applying second electromagnetic signals.

### TECHNICAL FIELD

The present disclosure it directed to systems and methods for treating patient hypertonicity, including patient spasticity and/or rigidity.

### BACKGROUND

A wide variety of mental and physical processes are known to be controlled or influenced by neural activity in particular regions of the brain. In some areas of the brain, such as in the sensory or motor cortices, the organization of the brain resembles a map of the human body; this is referred to as the “somatotopic organization of the brain.” There are several other areas of the brain that appear to have distinct functions that are located in specific regions of the brain in most individuals. For example, areas of the occipital lobes relate to vision, regions of the left inferior frontal lobes relate to language in the majority of people, and regions of the cerebral cortex appear to be consistently involved with conscious awareness, memory, and intellect. This type of location-specific functional organization of the brain, in which discrete locations of the brain are statistically likely to control particular mental or physical functions in normal individuals, is herein referred to as the “functional organization of the brain.”

Many problems or abnormalities with body functions can be caused by damage, disease and/or disorders of the brain. A stroke, for example, is one very common condition that damages the brain. Strokes are generally caused by emboli (e.g., obstructions of a vessel), hemorrhages (e.g., ruptures of a vessel), or thrombi (e.g., clotting) in the vascular system of a specific region of the cortex, which in turn generally cause a loss or impairment of a neural function (e.g., neural functions related to face muscles, limbs, speech, etc.). Stroke patients are typically treated using physical therapy to rehabilitate the loss of function of a limb or another affected body part. For most patients, little can be done to improve the function of the affected limb beyond the recovery that occurs naturally without intervention. One existing physical therapy technique for treating stroke patients constrains or restrains the use of a working body part of the patient to force the patient to use the affected body part. For example, the loss of use of a limb is treated by restraining the other limb. Although this type of physical therapy has shown some experimental efficacy, it is expensive, time-consuming and little-used. Stroke patients can also be treated using physical therapy plus other adjunctive therapies. For example, some types of drugs, such as amphetamines, that increase the activation of neurons in general, appear to enhance neural networks; these drugs, however, have limited efficacy because they are very non-selective in their mechanisms of action and cannot be delivered in appropriate concentrations directly at the site where they are needed. Therefore, there is a need to develop effective treatments for rehabilitating stroke patients and patients who have other types of brain damage.

The neural activity in the brain can be influenced by electrical energy that is supplied from an external source outside of the body. Various neural functions can thus be promoted or disrupted by applying an electrical current to the cortex or other region of the brain. As a result, the quest for treating damage, disease and disorders in the brain has led to research directed toward using electricity or magnetism to control brain functions.

One promising type of treatment is to electrically stimulate cortical tissue with one or more implanted electrodes. These electrodes are typically placed epidurally or subdurally within the patient's skull at a cortical location selected to provide a benefit to the patient. This technique has been shown to be effective for addressing several motor dysfunctions, either alone or in combination with a behavioral therapy (e.g., a physical therapy) regimen. One potential challenge with this technique is that the patient's dysfunction may sometimes result in muscle spasticity and/or rigidity, in addition to a more obvious primary symptom, such as the loss of limb use. While the spasticity and/or rigidity may not be as severe as the primary motor dysfunction symptom, it can interfere with behavioral therapy. In other cases, the spasticity and/or rigidity by itself may be debilitating, or at least present a significant hindrance to the patient.

One technique for addressing spasticity and/or rigidity is to identify and resect the brain tissue responsible for these motor dysfunctions. A drawback with this technique is that it is highly invasive and irreversible. Accordingly, other techniques, including drug-based therapies, have also been developed. However, these techniques can have additional drawbacks, including undesirable or inconvenient loss of effect over time (e.g., as a drug such as Botox wears off), side effects (e.g., if delivered orally), surgical risk (e.g., if delivered to the spinal cord by an intrathecal pump), and/or irreversibility (e.g., if phenol, botox or a similar drug is delivered directly to the muscle). Accordingly, there is a need for improved techniques for handling patient spasticity and/or rigidity.

### BRIEF DESCRIPTION OF THE DRAWINGS

FIG. 1A is a schematic illustration of selected neurons.

FIG. 1B is a graph illustrating the firing of an “action potential” associated with normal neural activity.

FIG. 2 is a flow diagram illustrating a method for addressing patient hypertonicity with electromagnetic signals in accordance with an embodiment of the invention.

FIG. 3 is a top plan view of a portion of the brain illustrating a target neural population selected in accordance with an embodiment of the invention.

FIG. 4 is a top plan view of a portion of the brain illustrating a target neural population selected in accordance with another embodiment of the invention.

FIG. 5 is a flow diagram illustrating a method for providing multiple signals to a patient in accordance with multiple signal delivery parameters.

FIG. 6A is a top plan view of a portion of the brain illustrating multiple target neural populations selected in accordance with an embodiment of the invention.

FIG. 6B is a graph illustrating firing an “action potential” associated with neural activity affected by a method in accordance with an embodiment of the invention.

FIGS. 7A and 7B are schematic illustrations of an implanting procedure in accordance with an embodiment of the invention.

FIG. 8 is an isometric illustration of an implantable signal delivery device configured in accordance with an embodiment of the invention.

FIG. 9 is a cross-sectional view schematically illustrating an implantable signal delivery device configured in accordance with an embodiment of the invention.

FIG. 10 illustrates a system for providing therapy to a patient in accordance with an embodiment of the invention.

FIG. 11 is a top plan view of a portion of the brain with a signal delivery device positioned in accordance with another embodiment of the invention.

FIG. 12A is a top, partially hidden isometric view of a signal delivery device configured in accordance with another embodiment of the invention.

FIG. 12B is an internal block diagram of a signal delivery device configured in accordance with yet another embodiment of the invention.

FIG. 13 is a flow diagram illustrating a method for treating a patient in accordance with still another embodiment of the invention.

### DETAILED DESCRIPTION

### A. Introduction

The following disclosure describes several methods and systems for treating patient hypertonicity. As used herein, hypertonicity refers generally to dysfunctional muscle tightness, and includes spasticity and/or rigidity. An aspect of several methods and systems in accordance with embodiments of the invention is to provide electromagnetic stimulation that addresses patient hypertonicity, either as a standalone treatment, or as part of a treatment that includes additional stimulation, for example, to enhance, facilitate, and/or otherwise improve a related or a different patient condition or functional ability. In some situations, an improvement in a patient condition or functional ability may correspond to a neuroplastic effect associated with one or more targeted neural structures.

One method for treating a patient includes identifying or estimating the location of a target neural population at, within, or having projections to the cortex of the brain associated with patient hypertonicity, and reducing or eliminating the patient's hypertonicity for a given time period by applying or directing electromagnetic signals to the target neural population. In particular embodiments, the electromagnetic signals can be applied to a cortical structure from an implanted signal delivery device, and can trigger signals that propagate to a sub-cortical structure, e.g., an alpha motor neuron, which is expected to be inhibited by stimulation of the cortical structure.

Another method can include identifying a target area of the central nervous system associated with hypertonicity, and then reducing or eliminating hypertonicity by applying first electromagnetic signals to the target area in accordance with a first set of signal delivery parameters. The method can further include applying second electromagnetic signals in accordance with a second set of signal delivery parameters different than the first set to affect, reduce, or eliminate an additional patient dysfunction. In a particular embodiment, the second electromagnetic signals can be applied to an area of the central nervous system where a change in an intrinsic neural-activity is suspected of occurring to carry out a particular physical function, and/or cognitive or other function (e.g., a neuroplastic region). Depending upon the nature and/or extent of a patient's neurologic dysfunction and/or embodiment details, one or more target neural populations to which electromagnetic signals are directed for addressing hypertonicity may be the same as, generally the same as, or different from a set of target neural populations to which electromagnetic signals are directed for addressing another patient condition, symptom, or functional impairment.

In further particular aspects, the method can also include engaging the patient in an adjunctive behavioral therapy, for example, a physical therapy, a cognitive therapy, a role-playing therapy, a language therapy, an auditory therapy (e.g., music or rhythm-based therapy or a tone discrimination task), and/or other therapy. One or more of such therapies may involve patient interaction with a mechanical, electronic, or computer-based device such as a keyboard, a mouse, a touch screen, a virtual reality device, an electronic drawing device (e.g., a stylus and digitizing tablet), or other type of user interface. Hence, patient performance during a behavioral therapy, and the extent to which the patient achieves functional gains in association with the behavioral therapy, may be enhanced by a reduction in patient hypertonicity. In certain aspects, the method may additionally or alternatively include administering an adjunctive chemical substance therapy (e.g., a spasticity reduction drug) to the patient (e.g., at particular times of day, or before, during, and/or after a behavioral therapy).

As an example, the first electromagnetic signals can be applied before the patient engages in the behavioral therapy to reduce and/or eliminate patient hypertonicity, after which the patient may proceed with the behavioral therapy. The second electromagnetic signals may be applied while engaging the patient in the behavioral therapy, for example, to enhance and/or facilitate neuroplasticity. The first electromagnetic signals may (optionally) also be applied during the behavioral therapy, for example, at pre-selected time intervals, and/or in response to an indication (e.g., an observed or measured indication) that the effect of a prior application of such signals has diminished.

In various situations, a reduction in hypertonicity achieved in association with an application of the first electromagnetic signals may persist, linger, or at least temporarily remain after delivery of the first electromagnetic signals is interrupted or terminated. For instance, an overall reduction in hypertonicity may last for several seconds, several minutes, or an hour or more after the application of the first electromagnetic signals. A maximal degree or level of hypertonicity reduction may occur during or following the application of the first electromagnetic signals. Following the cessation or interruption of the first electromagnetic signals, an extent to which the patient's hypertonicity is reduced may progressively decrease, such that the level of hypertonicity shifts or increases toward or returns to a baseline level.

A patient treatment system in accordance with still further aspects can include a signal delivery device configured to deliver electromagnetic signals to a patient, a sensor coupleable to the patient and configured to detect an indication of incipient or actual patient hypertonicity, and a controller coupled to the signal delivery device and/or the sensor. The controller can receive sensor signals corresponding to the indication of hypertonicity, and can direct or indicate the delivery of electromagnetic signals to the patient, based at least in part on the sensor signals. Accordingly, the system can operate in a feedback manner to provide therapeutic electromagnetic signals in response to an indication of hypertonicity.

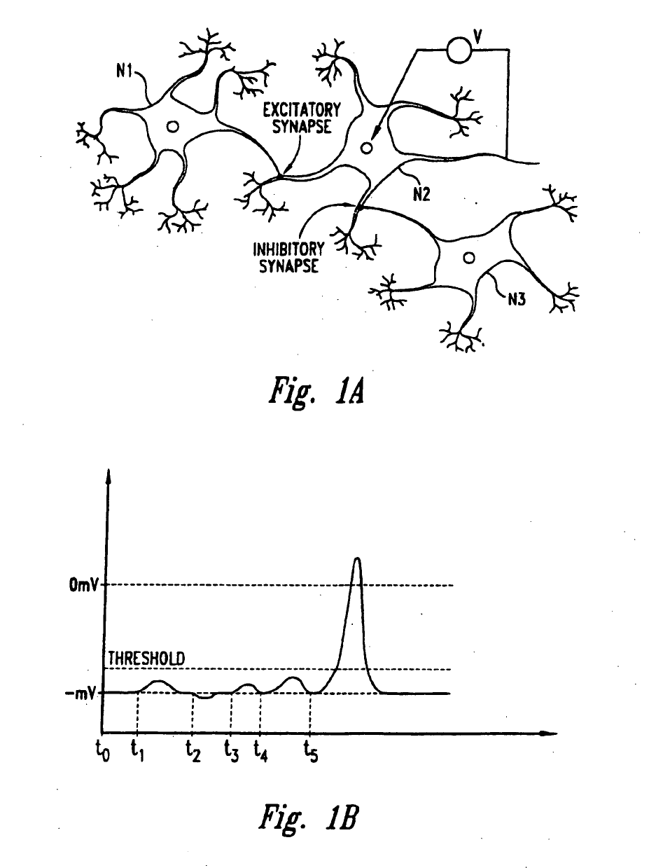

Several embodiments of systems and methods for treating patient hypertonicity are described below. A person skilled in the relevant art will understand, however, that the invention may have additional embodiments, and that the invention may be practiced without several of the details of the embodiments described below with reference to FIGS. 1A-13.

### B. Representative Methods and Systems

FIG. 1A is a schematic representation of several neurons N1-N3 and FIG. 1B is a graph illustrating an “action potential” related to neural activity in a normal neuron. Neural activity is governed by electrical impulses generated in neurons. For example, neuron N1 can send excitatory inputs to neuron N2 (e.g., at times t₁, t₃ and t₄ in FIG. 1B), and neuron N3 can send inhibitory inputs to neuron N2 (e.g., at time t₂ in FIG. 1B). The neurons receive/send excitatory and inhibitory inputs from/to a population of other neurons. The excitatory and inhibitory inputs can produce “action potentials” in the neurons, which are electrical pulses that travel through neurons by changing the flux of sodium (Na) and potassium (K) ions across the cell membrane. An action potential occurs when the resting membrane potential of the neuron surpasses a threshold level. When this threshold level is reached, an “all-or-nothing” action potential is generated. For example, as shown in FIG. 1B, the excitatory input at time t₅ causes neuron N2 to “fire” an action potential because the input exceeds the threshold level for generating the action potential. The action potentials propagate down the length of the axon (the long process of the neuron that makes up nerves or neuronal tracts) to cause the release of neurotransmitters from that neuron that will further influence adjacent neurons.

Patient hypertonicity may occur when a set of motor neurons are overactivated and accordingly overdrive the muscles with which it is associated. The results can include muscle rigidity and/or spasticity. Accordingly, it is believed that reducing the net excitatory input received by the subject motor neurons can reduce hypertonicity. In a particular embodiment, upper motor neurons (e.g., neurons located at or near the cortex of the brain) having an inhibitory synaptic association with lower motor neurons (e.g., alpha motor neurons) are directly and/or indirectly electrically stimulated via cortically applied signals (e.g., via a cortical implant). By stimulating a target population of upper motor neurons, and/or stimulating a target population of neurons having excitatory or inhibitory connections or projections to particular upper motor neurons, it is expected that the lower motor neurons will be inhibited and the patient's hypertonicity reduced or eliminated.

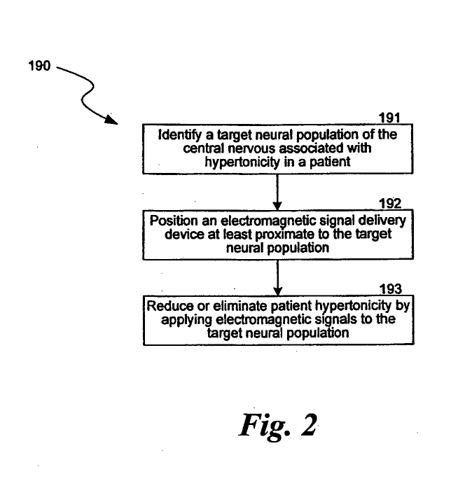

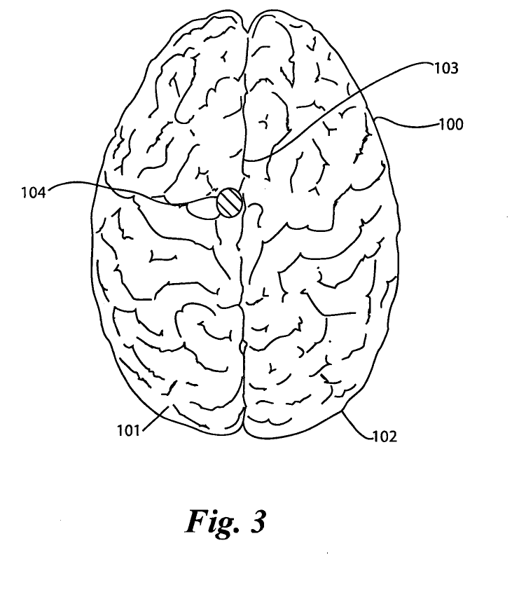

FIG. 2 is a flow chart illustrating a representative process 190 for treating a patient in accordance with an embodiment of the invention. The process 190 can include identifying at least one target neural population of the central nervous system associated with hypertonicity in a patient (process portion 191). For example, process portion 191 can include identifying neurons located at the cortex of the brain, which, when activated, can have an inhibitory effect on lower motor neurons. Process portion 192 can include positioning an electromagnetic signal delivery device at least proximate to the target neural population. For example, process portion 192 can include positioning one or more electrodes proximate to the target neural population. In particular embodiments, the electrodes are implanted within the patients' skull at an epidural or subdural cortical location. In process portion 193, the patient's hypertonicity can be reduced or eliminated by applying electromagnetic signals to the target neural population, via the signal delivery device. For example, the signals can be delivered continuously and/or on an as-needed basis, and/or in conjunction with other treatments and/or treatment parameters. Further details of each of the processes shown in FIG. 2 are described in greater detail below with reference FIGS. 3-13.

FIG. 3 is a top plan view of a patient's brain 100, illustrating the left lobe 101, the right lobe 102, and the central sulcus 103 between the left and right lobes 101, 102. In this particular example, the patient may suffer from forearm rigidity, affecting the elbow and/or wrist of the patient's right arm. The practitioner can accordingly select one or more target neural populations 104 based on the known somatotopic organization of the cortex, to locate a target neural population within a region of the primary motor cortex, the premotor cortex, and/or the supplementary motor area (SMA) generally associated with the function of the forearm and/or hand (e.g., the elbow and/or the wrist). The practitioner can select the target neural population 104 with reference to known anatomical structures, including the central sulcus 103 and other gyri and sulci located relative to (e.g., laterally from) the central sulcus 103. If the patient's hypertonicity affects other motor functions, the practitioner can rely on a generally similar technique (and other known anatomical structures) to select an appropriate target neural population.

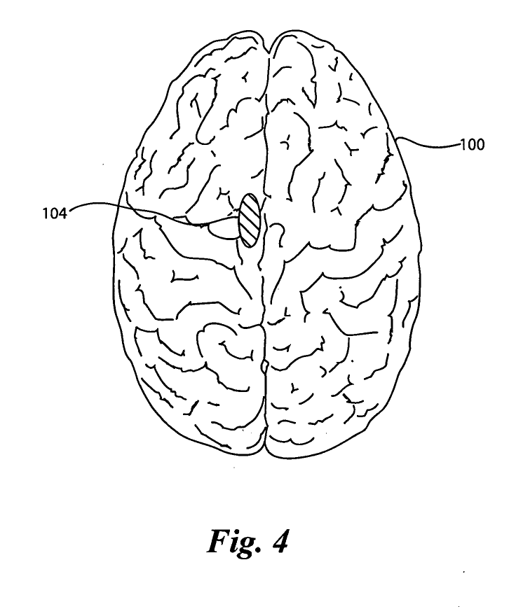

In other embodiments, the practitioner may wish to rely on information in addition to or in lieu of anatomical landmarks. For example, in some cases, the practitioner may wish to locate the target neural population with more precision than may be practicable by relying on anatomical landmarks alone. In other cases, the patient's hypertonicity may not necessarily be associated with cortical structures that one would expect, based on the usual somatotopic organization of the brain. For example, in some cases, portions of a neural structure usually associated with a particular motor function may be damaged. Another area of the brain may attempt to at least partially take over that function, and in the process, may generate hypertonicity. In such instances, the practitioner may use neurofunctional localization techniques (e.g., neural imaging techniques) to locate the appropriate target neural population 104. Referring now to FIG. 4, the practitioner may use fMRI to generate a color-coded map of the brain 100 that highlights a target neural population 104 as one that is primarily active and/or responsible for patient hypertonicity. Suitable fMRI results typically require a comparison of the patient's brain at two different conditions, for example, one condition at which spasticity is present and another at which it is reduced or absent. Accordingly, the image shown in FIG. 4 can be generated by comparing a suitable imaging parameter (e.g., cerebral blood flow or oxygenation levels) at two conditions: one with the patient engaging or attempting to engage in a task that results in an expression of hypertonicity, and another with the patient engaging in a task that does not. In a further particular example, if the patient's forearm is rigid and the patient's fingers are not, the patient can be asked to perform a task that involves both the forearm and the fingers (eliciting both a hypertonic response and a normal response), and then perform a task that involves only the fingers (eliciting only a normal response). By subtracting the results of the latter test from the former, the practitioner can isolate the area of the brain responsible for the hypertonic behavior of the forearm. A comparison between brain areas under two conditions may additionally or alternatively involve the administration of a drug to the patient, for example, to facilitate comparison of a baseline hypertonic state with a drug-related reduced hypertonicity state.

In other embodiments, other techniques can be used to locate a target neural population associated with patient hypertonicity. For example, diffusion tensor imaging (DTI) can be used to identify an appropriate set of descending neural projections to which electromagnetic signals may be directed. More specifically, the practitioner can identify a “seed point” at the motor cortex or another brain location involved in motor function (e.g., the premotor cortex or the SMA) using known anatomic information, or fMRI-based data. Using the seed point, the practitioner can perform a fiber tracking analysis to identify fibers that connect the primary motor cortex to the spinal motor tracts. This technique can be performed for multiple seed points, and the target neural population can then be selected to include the area(s) of the cortex that corresponds to a neural path having the highest density of (intact) fiber tracts. In still further embodiments, diffusion weighted imaging can be used to identify areas of increased anisotropy, which may correspond to an increased number of intact neurons.

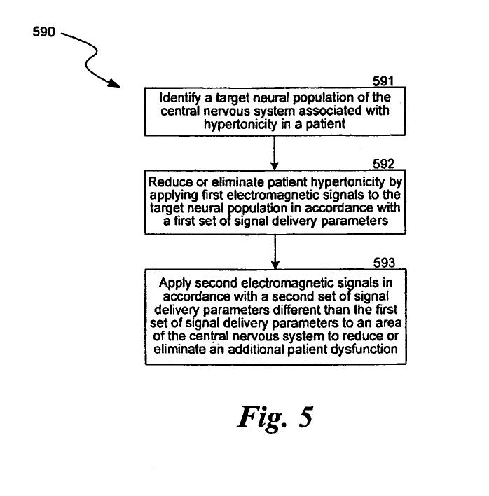

In the examples described above, the practitioner can address patient hypertonicity in a standalone manner. In other embodiments, the patient's hypertonicity may interfere with other, potentially related therapies that the practitioner wishes to carry out. For example, the patient's hypertonicity may interfere with efforts to engage the patient in a behavioral therapy, which in turn, forms part of a treatment regimen for addressing a related or different motor dysfunction. In a particular example, the patient may have suffered neurologic damage (e.g., as a result of a stroke or traumatic brain injury), which causes the patient to lose the ability to effectively move his or her fingers. To address particular effects or symptoms resulting from the neurologic damage, the practitioner may wish to electromagnetically stimulate neural tissue that, by virtue of the electromagnetic stimulation, may be encouraged to take over or compensate for the function of damaged tissue. However, the patient's hypertonicity may interfere with the behavioral therapy aspects of this treatment regimen. Accordingly, other embodiments can include combining a treatment for addressing the patient's hypertonicity with treatments directed to addressing another dysfunction (e.g., a stroke-related motor dysfunction). A representative example is shown in FIG. 5.

FIG. 5 illustrates a representative process 590 for providing treatment to a patient. The process can include identifying a target neural population of the central nervous system (e.g., the brain cortex) associated with hypertonicity in a patient (process portion 591). Process portion 592 can include reducing or eliminating patient hypertonicity by applying first electromagnetic signals to the target neural population in accordance with a first set of signal delivery parameters. Process portion 593 can include applying second electromagnetic signals in accordance with a second set of signal delivery parameters different than the first set of signal delivery parameters. The second electromagnetic signals can be directed to an area of the central nervous system where the expected effect is to reduce or eliminate another patient dysfunction. For example, in a particular embodiment, the second electromagnetic signals can be directed to a location where a change in an intrinsic neural activity is suspected of occurring to carry out a particular physical function and/or cognitive function (e.g., a neuroplastic region). This approach may be used to address motor, cognitive, mood, sensory, and/or other dysfunctions, including those associated with stroke. Such dysfunctions may be associated with other patient conditions as well, e.g., multiple sclerosis or Parkinson's Disease. The dysfunctions may be associated with the intrinsic patient condition (e.g., multiple sclerosis or Parkinson's Disease) and/or with side effects resulting from drug-based or other treatments of the intrinsic condition.

The first and second signal delivery parameters can include the location of the applied signals, as well as the characteristics with which the applied signals are delivered to the patient. For example, the second electromagnetic signals can be applied to the same target neural population as the first electromagnetic signals, or they can be applied to a different population. The polarity with which the first and second electromagnetic signals are applied can also be the same or different, depending upon the specific application. For example, the first electromagnetic signals can be anodal unipolar signals, and the second electromagnetic signals can be cathodal. It is generally expected that anodal signals will have an inhibitory effect on patient hypertonicity, with lower current and/or voltage levels than would be required for cathodal stimulation. Accordingly, it is expected that anodal stimulation will be more likely than cathodal stimulation to address patient hypertonicity in an efficient manner. Conversely, cathodal stimulation is generally expected to have a more beneficial effect than anodal stimulation in the context of facilitating patient neuroplasticity. Accordingly, the practitioner can select a signal polarity appropriate for the therapeutic task at hand. If the same target neural population is to receive signals addressing both hypertonicity and neuroplasticity, the patient can receive alternating first and second signals. In some embodiments, the first and/or the second electromagnetic signals may be bipolar signals.

In other embodiments, signal delivery parameters other than polarity and/or location can be varied. For example, the current, voltage, frequency, pulse width, interpulse interval and/or bursting pattern of the signals can be different for the first signals than for the second signals. Representative examples of systems and methods for applying signals in accordance with multiple parameter sets are included in co-pending U.S. application Ser. No. 11/183,713, filed on Jul. 15, 2005 and incorporated herein by reference. In any of these embodiments, the first signal delivery parameters can be selected to address patient hypertonicity, and the second signal delivery parameters can be selected to address neuroplasticity and/or other neural functions.

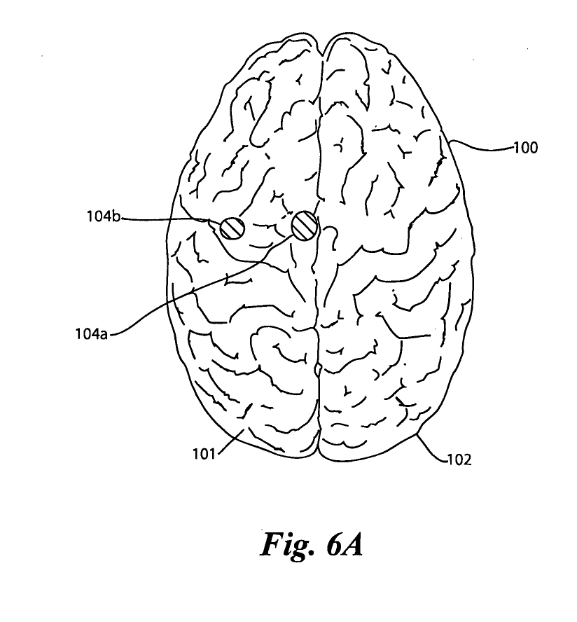

FIG. 6A illustrates the brain 100 with a first target neural population 104a and a second target neural population 104b both identified. The first target neural population 104a can be one that is associated with the motion of the patient's forearm, and the second target neural population 104b can be associated with motion of the patient's fingers. Accordingly, both areas can be stimulated in the same, similar, and/or different manners to address patient dysfunction involving both areas. As discussed in an example above, the first target neural population 104a may be stimulated to address patient hypertonicity, and the second target neural population 104b may be stimulated in a manner to facilitate the patient's natural neuroplastic response. In an embodiment shown in FIG. 6A, the first and second neural populations 104a, 104b are shown in the same brain hemisphere. In other embodiments, the first target neural population 104a may be in a different hemisphere than is the second target neural population 104b. In still further embodiments, the patient may be stimulated at multiple first target neural populations 104a, which may be located in one or both hemispheres, and/or at multiple second target neural populations 104b, which may be located in one or both hemispheres. Accordingly, particular embodiments can include providing signals to any of a variety of combinations of first and second neural populations, located in either hemisphere or both hemispheres, depending upon the particular patient.

As discussed initially above, one or more characteristics of signals (e.g., the first signals) applied to address hypertonicity may be different than the characteristics of signals (e.g., the second signals) applied to facilitate neuroplasticity. One way in which the two types of signals may differ is associated with how close the signals come to triggering an action potential. The dendrites of any given neuron continually receive excitatory and inhibitory input signals from other neurons to which the dendrites are synaptically connected. In response to the excitatory and inhibitory inputs, the dendrites generate descending depolarization waves. Within the neuron, the descending depolarization waves are summated or integrated. When the magnitude of this summation exceeds a threshold firing level, the neuron generates or “fires” an action potential, which propagates along the neuron's axon to synapses in communication with other neurons.

Extrinsic stimulation signals (e.g., electrical stimulation signals applied via an electrode) may be viewed as having a modulatory effect upon the excitatory and inhibitory input signals that the dendrites within a target neural population receive. In particular, electrical stimulation signals may alter the distribution of mobile ions along neural membranes, and/or affect voltage-gated ion channels within the neuron. The presence and characteristics of extrinsic stimulation signals can affect the likelihood that a population of neurons will generate a sufficient number of action potentials to trigger an associated neural function (e.g., a movement).

FIG. 6B is a graph illustrating the application of a subthreshold potential to the neurons N1-N3 initially shown in FIG. 1A. At times t₁ and t₂, the depolarization waves generated in response to the intrinsic excitatory/inhibitory inputs from other neurons do not summate in a manner that “bridges-the-gap” from a neural resting potential at −X mV (e.g., approximately −70 mV) to a threshold firing potential at −T mV (e.g., approximately −50 mV). At time t₃, extrinsic electrical stimulation is applied to the brain, in this case at an intensity or level that is expected to augment or increase the magnitude of descending depolarization waves generated by the dendrites, yet below an intensity or level that by itself will be sufficient to summate in a manner that induces action potentials and triggers the neural function corresponding to these neurons. Extrinsic stimulation signals applied in this manner may generally be referred to as subthreshold signals. At time t₄, the neurons receive another excitatory input. In association with a set of appropriately applied extrinsic stimulation signals, even a small additional intrinsic input may result in an increased likelihood that a summation of the descending depolarization waves generated by the dendrites will be sufficient to exceed the difference between the neural resting potential and the threshold firing potential to induce action potentials in these neurons. Thus, in this situation, the subthreshold extrinsic signals facilitate the generation of action potentials in response to intrinsically occurring neural signaling processes. It is to be understood that depending upon signal parameters, the extrinsic signals may exert an opposite (disfacilitatory, inhibitory, or disruptive) effect upon neurons or neural signaling processes, and hence particular signal parameters may be selected in accordance with a likelihood of achieving a desired or intended therapeutic effect or outcome at any given time.

The actual signal(s) applied by one or more extrinsic signal delivery devices positioned in, upon, or above the brain to achieve a therapeutic or intended effect will vary according to the individual patient, the type of therapy, the type of electrodes, and/or other factors. In general, the pulse form(s) of the first and/or second electromagnetic signals (e.g., the frequency, pulse width, waveform, current level, and/or voltage) directed toward achieving an intended therapeutic effect may be selected or estimated relative to a test signal level or intensity at which a neural function is triggered or activated, or a change in a physiologic parameter (e.g., cerebral blood flow) is detected. Additionally or alternatively, the pulse form(s) of the first and/or second electromagnetic signals may be selected, adjusted, modulated, limited, or constrained at one or more times relative to parameters corresponding to one or more previously (e.g., most-recently) applied signals, or a maximum allowable or intended peak or average stimulation signal intensity.

A set of test signals may be applied as part of a threshold test procedure during which test signal parameters are modified (e.g., a current or voltage level is increased, or a pulse width is increased) until a patient response or state change is measured, detected, observed, or reported. A patient response may correspond to, for example, a patient movement, a patient sensation, or the presence of or change in a physiologic or physiologic correlate measure such as a motor evoked potential (MEP) signal, an electroencephalograph (EEG) or electrocorticograph (ECOG) signal, or a hemodynamic parameter. A test signal intensity or level that results in a given type of patient response may be defined as a patient response threshold, or more generally as a threshold level or difference.

The particular therapeutic signal level selected by the practitioner can depend on whether the signal is intended to address hypertonicity (e.g., via a first signal) or another condition (e.g., via a second signal). The first signal can have one or more values in the range of from about 70% to about 95% of a patient response threshold level, possibly up to a maximum desirable or allowable peak, average, or cumulatively defined level. In some cases, the first signals can be suprathreshold or essentially suprathreshold, though it is expected that subthreshold signals will reduce system power consumption, and will have a lower likelihood of saturating the target neural population. The second signal can have one or more values in the range of from about 25% to about 75% (e.g., about 50%) of a patient response threshold level, which may also be defined relative to a maximum desirable or allowable level. The second signal may (but need not) be selected to facilitate a neuroplastic response.

In some situations, the first signal may be applied in a manner that corresponds to a first type of patient response threshold, and the second signal may be applied in a manner that corresponds to a second type of patient response threshold. For example, the first signal may be applied at a level that corresponds to a first type of movement, or a first body part or a first bodily function, and the second signal may be applied at a level that corresponds to a second type of movement or a second body part or a second bodily function. As another example, the first signal may be applied at a level that corresponds to a movement, and the second signal may be applied at a level that corresponds to a test signal effect upon a sensation, a neuropsychiatric or neurocognitive task performance (e.g., a working memory task), or a change in an electroencephalographic or hemodynamic parameter.

In one embodiment, neural stimulation efficacy may be sustained or improved through the application or delivery of one or more suprathreshold or near-suprathreshold pulses or bursts during a neural stimulation procedure that is primarily characterized by subthreshold stimulation. Such suprathreshold pulses or bursts may occur in a predetermined, aperiodic, or random manner. For example, during a subthreshold stimulation procedure that applies stimulation signals at a current level corresponding to approximately 50% of a movement, motor evoked potential (MEP), or sensation threshold, a suprathreshold pulse or pulse set may be applied at a current level corresponding to approximately 100% of such a threshold at regular intervals (e.g., once every x seconds or once every y minutes (e.g., once every 3, 10, 15, or 30 minutes)), or at random times that fall between a minimum and a maximum allowable length time period. The following additional examples illustrate further representative methods for treating patient hypertonicity.

### EXAMPLE 1

A patient receives unipolar (e.g. anodal unipolar) and/or bipolar stimulation at from about 70-90% (e.g., approximately 80%) of a movement threshold for approximately 2-30 minutes (e.g., about 5-20 minutes) to reduce rigidity. The patient then receives anodal, cathodal, and/or bipolar stimulation at from about 20-80%, 25-75%, or 30-60% (e.g., approximately 50%) of a patient response threshold (e.g., a movement or other threshold) during behavioral therapy. After a given period of time has elapsed during a behavioral therapy session (e.g., approximately 30-90 minutes, or approximately 60 minutes), the patient may receive a follow-up series of unipolar and/or bipolar pulses at 70-90% of a movement threshold to reduce rigidity or maintain an acceptable level of rigidity. A computer or programming device can notify the patient or a practitioner when a rigidity treatment session should begin or end. The duration and/or intensity of successive rigidity treatment sessions may be identical or different. Additionally, stimulation signals may be applied at or between one or more levels or intensities relative to an acceptable range (e.g., between approximately 70-95% of an MEG, movement, or other threshold for addressing hypertonicity; or 25-75% of a patient response threshold for addressing functional development/recovery) based upon signal polarity, stimulation site (e.g., in an affected and/or unaffected hemisphere), and/or a type of patient response threshold under consideration.

### EXAMPLE 2

A patient receives anodal stimulation at approximately 80% of movement threshold for about 20 minutes, prior to receiving cathodal neural stimulation at 50% of a patient response threshold (e.g., a movement threshold) during physical therapy. After the patient's rigidity has increased or returned to a given level, or once a task performance level or other hypertonicity measure has begun to wane or decrease, the patient may receive a follow-up rigidity treatment session (e.g., as described herein) before continuing additional or other behavioral therapy. The patient's rigidity or task performance level may be determined by a practitioner (e.g., based upon an electrophysiological measurement (e.g., and EMG measurement), the patient, or in association with an Ashworth-based or other clinical measure such as a reflex, coordination, or motion control test. The patient's hypertonicity or task performance level may additionally or alternatively be determined by an automated or semiautomated system, in which case a computer or programming device can generate an alert or notification indicating that a rigidity treatment session may be beneficial. For instance, a computer coupled to a mouse and/or a drawing tablet may include program instructions that monitor or evaluate patient performance on a drawing test such as that described by Eder et al. in “The drawing test: assessment of coordination abilities and correlation with clinical measurement of spasticity,” Arch. Phys. Med. Rehabil. 2005 February; 86(2):289-295, incorporated herein by reference in its entirety.

### EXAMPLE 3

A patient receives anodal stimulation at approximately 80% of movement threshold for 20 minutes prior to receiving cathodal neural stimulation at 50% of movement threshold during physical therapy. After the patient's rigidity has increased or returned to a given level, or once a task performance level has begun to wane or decrease, the patient may receive brief, occasional, or periodic pulses or pulse bursts at 70-80% of movement threshold (e.g., a brief burst at 80% of movement threshold every 10-20 minutes) as the physical therapy session continues. Again, rigidity or task performance may be monitored by a practitioner and/or an automated system.

### EXAMPLE 4

A patient receives anodal stimulation at approximately 80% of movement threshold for 20 minutes prior to receiving cathodal neural stimulation at 50% of movement threshold during behavioral therapy. During behavioral therapy, the patient receives automatically delivered periodic bursts of anodal stimulation at approximately 80% of movement threshold, intermixed with cathodal stimulation at approximately 50% of movement threshold. The periodic bursts of anodal stimulation are expected to prevent, reduce, or delay functional performance degradation.

### EXAMPLE 5

A patient receives anodal or cathodal stimulation at 70-90% of movement threshold to reduce hypertonicity. An automated device (e.g., an EMG device configured to monitor patient H-waves), and/or the practitioner, determines the length of time that patient hypertonicity is reduced, as a result of the stimulation. During physical therapy, cathodal stimulation is applied to the patient at about 50% of movement threshold, and is intermixed with anodal or cathodal stimulation at 70-90% of movement threshold at intervals corresponding to the determined length of time. Results obtained from one or more length-of-time determinations can be saved for later use. These results can be used to provide stimulation to the patent during and/or outside a physical therapy session.

### EXAMPLE 6

A patient receives anodal, cathodal, and/or bipolar stimulation at 70-90% of movement threshold to reduce hypertonicity. During physical therapy, the patient receives cathodal stimulation at about 50% of movement threshold. Also during physical therapy an automated device (e.g., an EMG device configured to monitor patient H-waves) detects the onset and/or incipiency of hypertonicity and triggers the delivery of anodal or cathodal stimulation at 70-90% of movement threshold. The arrangement can also be used outside physical therapy. It is expected that this arrangement may have particular benefit when it is difficult to determine how long the hypertonicity signals last, and/or when hypertonicity increases as a result of the patients' intention to move (as may occur during and outside a physical therapy session). In a further particular example, the patient receives anodal or cathodal stimulation at 70-90% of a movement or other threshold to reduce hypertonicity, automatically, for example, while the patient is sleeping, e.g., at a selected time prior to the patient's expected waking time. It is expected that this technique can reduce patient hypertonicity after the patient awakens.

### EXAMPLE 7

A patient receives ipsilesional anodal stimulation at approximately 80% of movement threshold for 20 minutes to reduce/alleviate rigidity; followed by at least one type of neural stimulation directed toward functional development/recovery that is applied to one or both brain hemispheres, including during a portion of at least one behavioral therapy session. The neural stimulation directed toward functional development/recovery may be applied at approximately 25-75% (e.g., about 50%) of a patient response threshold, or at a magnitude or intensity below a maximum allowable level, possibly depending upon a type of neural stimulation or behavioral therapy under consideration at any given time. Additional or subsequent stimulation for treating hypertonicity may be delivered in one or more manners described herein. In this example, ipsilesional stimulation refers to stimulation applied to the brain hemisphere in which the cortical structures expected to affect hypertonicity are located.

### EXAMPLE 8

A patient suffers impairment to the right fingers, for example, due to stroke, traumatic brain injury, or other cause. The patient also suffers from spasticity/rigidity affecting the right shoulder. The patient receives stimulation at the left hemisphere motor cortex (at or near the brain portion expected to control right should motor function), at approximately 80% of movement threshold to reduce/alleviate spasticity/rigidity. The patient also receives (simultaneously and/or sequentially) inhibitory stimulation at the right hemisphere motor cortex (at or near the brain portion expected to control finger function) to discourage recruitment of right side neurons to take over the function of (defective) left side neurons. This stimulation can be monopolar or bipolar, periodic or aperiodic. Optionally, the patient can also receive facilitatory stimulation at the left hemisphere (at or near the brain portion expected to control right finger motor function), using different parameters than those used to control (a) spasticity/rigidity and/or (b) inhibition of right hemisphere neurons. For example, this optional stimulation can be less than 80% of motor threshold (e.g., 50% of motor threshold) and can be delivered at a different frequency than the inhibitory stimulation applied to the right hemisphere. The foregoing arrangement can (a) reduce/alleviate spasticity/rigidity, (b) address paradoxical facilitation and (c) promote a left brain neuroplastic response.

### EXAMPLE 9

Outside of supervised behavioral therapy sessions, a patient uses a patient programmer one or more times per day (e.g., at predetermined or patient-selectable times) to activate a predefined rigidity treatment program. The rigidity treatment program can provide stimulation at, for example, approximately 70% of a most recently measured movement threshold. Alternatively, the rigidity treatment program may provide stimulation at one or more current levels, up to a maximum allowable level, possibly based upon time of day, a cumulative measure (e.g., a total time, or a total electrical current dose) of stimulation signals applied to the patient relative to a given time period (e.g., several hours or a day), and/or patient input that estimates or characterizes the patient's present rigidity condition.

### EXAMPLE 10a

To reduce/alleviate hypertonicity, a patient receives a first set of neural stimulation signals at a first set of stimulation sites at approximately 80-95% of a movement threshold, or at a maximum desirable or allowable level, for 2-20 minutes. The patient additionally (e.g., subsequently) receives a second set of stimulation signals in accordance with at least one type of neural stimulation directed toward lasting, long-term, or permanent functional development/recovery (e.g., achieved in association with neuroplasticity), which is applied to a second set of stimulation sites corresponding to one or both brain hemispheres during a portion of at least one behavioral therapy session. The neural stimulation directed toward functional recovery may be applied within a range of approximately 25-75% of a patient response threshold, or at a magnitude or intensity at or below a particular (e.g., maximum allowable) level. The second set of neural stimulation signals is primarily directed to addressing a non-hypertonicity patient dysfunction. Accordingly, in this example, the patient receives first stimulation signals directed to addressing acute hypertonicity, and second stimulation signals directed to achieving long-term reduction (via a neuroplasticity) of a non-hypertonicity patient dysfunction.

### EXAMPLE 10b

At one or more times during a behavioral therapy session, additional neural stimulation directed toward reducing hypertonicity, maintaining a reduced hypertonicity state, and/or facilitating a neuroplastic effect that may at least partially alleviate a hypertonic condition on a lasting, long-term, or permanent basis is applied at approximately 25-75% of a patient response threshold (e.g., movement, sensation, or MEP-based threshold), or relative to a maximum desirable or allowable level. For example, after applying the first set of stimulation signals, the second set of stimulation signals is applied to the first stimulation sites. The second set of stimulation signals may be interspersed or interleaved with the first set of stimulation signals. In other words, the first stimulation signals can address acute hypertonicity, and the second stimulation signals can be provided to utilize the neuroplastic effect for achieving a long-term reduction in hypertonicity.

### EXAMPLE 10c

In this example, aspects of Examples 10a and 10b are combined. In other words, the patient can receive first stimulation signals applied to the first stimulation site(s) to address acute hypertonicity, second stimulation signals applied to the second stimulation site(s) to achieve a long-term (e.g., neuroplasticity-based) reduction in a non-hypertonicity dysfunction, and third stimulation signals applied to the first stimulation site(s) to achieve a long-term (e.g., neuroplasticity-based) reduction in hypertonicity. The second and third sets of stimulation signals can follow the first stimulation signal. The second set of stimulation signals can be applied during or interspersed with the application with the third set of stimulation signals. For example, the second stimulation signals may be applied to the second stimulation site(s) approximately 80% of the time, and the third stimulation signals may be applied to the first stimulation site(s) approximately 20% of the time. The third stimulation signals may be applied at approximately 25-75% (e.g., 25%, 40-60%, or 65-70%) of a patient response threshold, or relative to a previously applied level, or a maximum allowable or desirable level. The third set of stimulation signals may be applied to the first set stimulation site(s) in accordance with at least one parameter that differs from the first and/or second sets of stimulation signals (e.g., at 25% of a movement threshold, or at a higher or lower pulse repetition frequency than that corresponding to the first or second sets of stimulation signals). Additionally or alternatively, the third set of stimulation signals may be applied outside of supervised behavioral therapy sessions, for example, in one or more manners previously described.

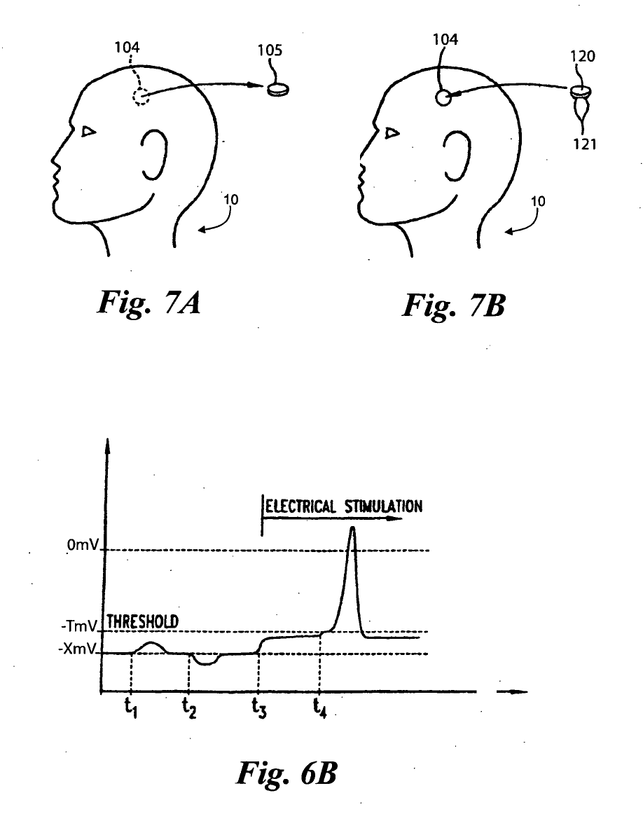

In any of the foregoing examples, the applied electromagnetic signals may be delivered by an implanted device. FIGS. 7A and 7B are schematic illustrations of an implanting procedure for positioning a signal delivery device at the brain of a patient P. Referring first to FIG. 7A, a skull section 105 is removed from the patient P adjacent to one or more target neural populations (a single target neural population 104 is shown in FIG. 7A for purposes of illustration). The skull section 105 can be removed by boring a hole in the skull 106 in a manner known in the relevant art, or a much smaller hole can be formed in the skull 106 using drilling techniques that are also known in the art. The hole can be 0.2-4.0 cm in diameter in a particular embodiment, but can have other dimensions depending upon factors that include the size (and/or number) of the target neural population(s), and/or the size of the implanted device.

Referring to FIG. 7B, an implantable signal delivery device 120 having first and second electrodes or contacts 121 can then be implanted in the patient P. Suitable techniques associated with the implantation procedure are known to practitioners skilled in the art. After the signal deliver device 120 has been implanted in the patient P, a pulse system generates electrical pulses that are transmitted to the target neural population 104 by the first and second electrodes 121.

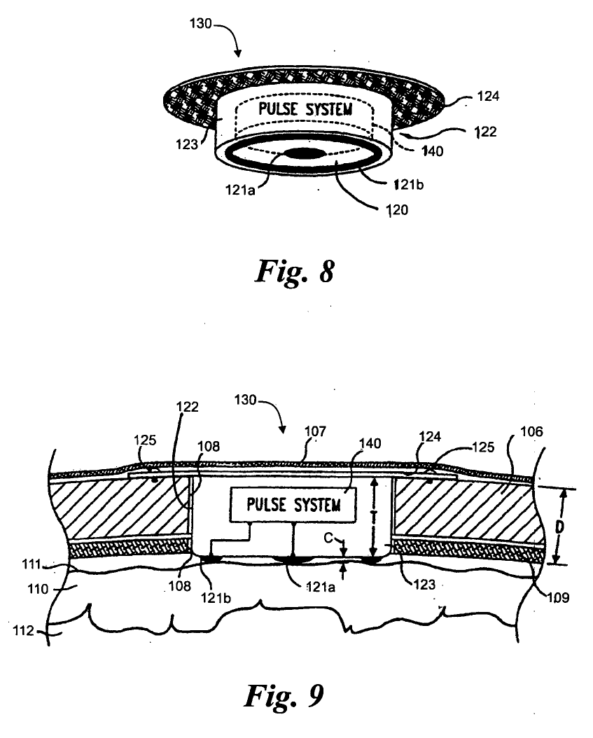

FIGS. 8-12B illustrate signal delivery devices configured in accordance with a variety of embodiments for providing electromagnetic signals to patients suffering from hypertonicity and/or other dysfunctions. Accordingly, these devices are representative of devices for performing the therapies described above. The illustrated devices include cranial implants that supply electrical current to the brain, as it is expected that such devices will provide direct treatment with relatively low power requirements. However, in other embodiments, other electromagnetic signals (e.g., magnetic fields) may be provided by other devices (e.g., transcranial magnetic stimulation devices).

FIG. 8 is an isometric view of a signal delivery system 130 configured in accordance with an embodiment of the invention for stimulating a region of the cortex proximate to the pial surface. The signal delivery system 130 can include an implantable signal delivery device 120 that in turn includes a support member 122, an integrated pulse system 140 (shown schematically) carried by the support member 122, and first and second electrodes 121 (identified individually by reference numbers 121a and 121b). The first and second electrodes 121 are electrically coupled to the pulse system 140. The support member 122 can be configured to be implanted into the skull or another intracranial region of a patient. In one embodiment, for example, the support member 122 includes a housing 123 and an attachment element 124 connected to the housing 123. The housing 123 can be a molded casing formed from a biocompatible material that has an interior cavity for carrying the pulse system 140. The housing 123 can alternatively be a biocompatible metal or another suitable material. The housing 123 can have a diameter of approximately 1-4 cm, and in many applications the housing 123 can be 1.5-2.5 cm in diameter. The housing 123 can also have other shapes (e.g., rectilinear, oval, elliptical) and other surface dimensions. The signal delivery system 130 can weigh 35 g or less and/or occupy a volume of 20 cc or less. The attachment element 124 can be a flexible cover, a rigid plate, a contoured cap, or another suitable element for holding the support member 122 relative to the skull or other body part of the patient. In one embodiment, the attachment element 124 is a mesh, such as a biocompatible polymeric mesh, metal mesh, or other suitable woven material. The attachment element 124 can alternatively be a flexible sheet of Mylar, a polyester, or another suitable material.

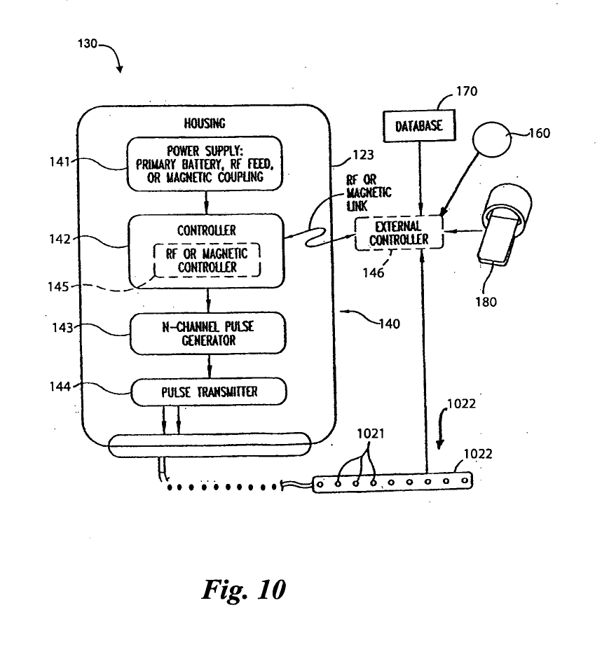

FIG. 9 illustrates a cross-sectional view of the signal delivery system 130 after it has been implanted into a patient in accordance with an embodiment of the invention. In this particular embodiment, the system 130 is implanted into the patient by forming an opening in the scalp 107 and cutting a hole 108 through the skull 106 and through the dura mater 109. The hole 108 should be sized to receive the housing 123 of the support member 122, and in most applications, the hole 108 should be smaller than the attachment element 124. A practitioner inserts the support member 123 into the hole 108 and then secures the attachment element 124 to the skull 106. The attachment element 124 can be secured to the skull using a plurality of fasteners 125 (e.g., screws, spikes, etc.) or an adhesive. In an alternative embodiment, a plurality of downwardly depending spikes can be formed integrally with the attachment element 124 to define anchors that can be driven into the skull 106.

The embodiment of the system 130 shown in FIG. 9 is configured to be implanted into a patient so that the electrodes 121 contact a desired portion of the brain at the stimulation site. The housing 123 and the electrodes 121 can project from the attachment element 124 by a distance “D” such that the electrodes 121 are positioned at least proximate to the pia mater 111 surrounding the cortex 110. The electrodes 121 can project from the housing 123 as shown in FIG. 9, or the electrodes 121 can be flush with the interior surface of the housing 123. In the particular embodiment shown in FIG. 9, the housing 123 has a thickness “T” and the electrodes 121 project from the housing 123 by a distance “C” so that the electrodes 121 apply a given amount of pressure against the surface of the pia mater 111. The thickness of the housing 123 can be approximately 0.5-4 cm, and is more generally about 1-2 cm. The configuration of the signal delivery system 130 is not limited to the embodiment shown in FIGS. 8 and 9, but rather the housing 123, the attachment element 124, and the electrodes 121 can be configured to position the electrodes 121 in several different regions of the brain. For example, in another embodiment, the housing 123 and the electrodes 121 can be configured to position the electrodes deep within the cortex 110, and/or a deep brain region 112.

The pulse system 140 shown in FIGS. 8 and 9 generates and/or transmits electrical pulses to the electrodes 121 to create an electrical field at the target neural population. The particular embodiment of the pulse system 140 shown in FIG. 9 is an “integrated” unit in that is carried by the support member 122. The pulse system 140, for example, can be housed within the housing 123 so that the electrodes 121 can be connected directly to the pulse system 140 without having leads outside of the signal delivery device 120. The distance between the electrodes 121 and the pulse system 140 can be less than 4 cm, and it is generally 0.10 to 2.0 cm. The system 130 can accordingly provide electrical pulses to the target neural population without having to surgically create tunnels running through the patient to connect the electrodes 121 to a pulse generator implanted remotely from the signal delivery device 120. It will be appreciated, however, that in other embodiments, the pulse system 140 can be implanted separately from the signal delivery device 120, within or outside the cranium.

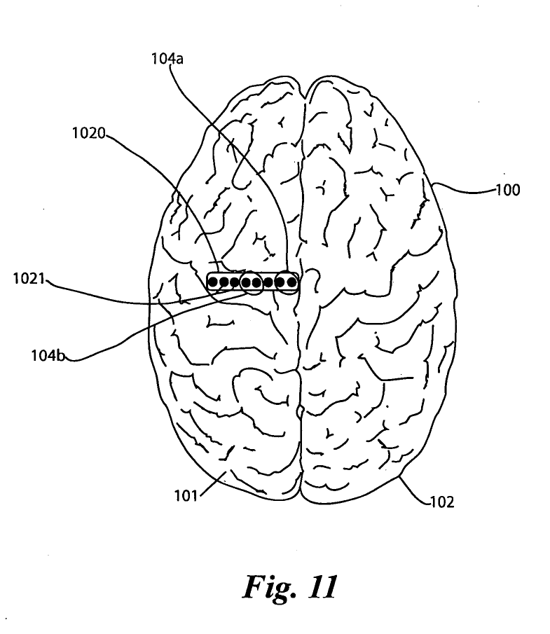

FIG. 10 schematically illustrates details of an embodiment of the pulse system 140 described above. The pulse system 140 is generally contained in the housing 123, which can also carry a power supply 141, an integrated controller 142, a pulse generator 143, and a pulse transmitter 144. In certain embodiments, a portion of the housing 123 may comprise a signal return electrode. The power supply 141 can comprise a primary battery, such as a rechargeable battery, or other suitable device for storing electrical energy (e.g., a capacitor or supercapacitor). In other embodiments, the power supply 141 can be an RF transducer or a magnetic transducer that receives broadcast energy emitted from an external power source and that converts the broadcast energy into power for the electrical components of the pulse system 140.

In one embodiment, the integrated controller 142 can include a processor, a memory, and/or a programmable computer medium. The integrated controller 142, for example, can be a microcomputer, and the programmable computer medium can include software loaded into the memory of the computer, and/or hardware that performs the requisite control functions. In another embodiment identified by dashed lines in FIG. 10, the integrated controller 142 can include an integrated RF or magnetic controller 145 that communicates with the external controller 146 via an RF or magnetic link. In such an embodiment, many of the functions performed by the integrated controller 142 may be resident on the external controller 146 and the integrated portion 145 of the integrated controller 142 may include a wireless communication system.

The integrated controller 142 is operatively coupled to, and provides control signals to, the pulse generator 143, which may include a plurality of channels that send appropriate electrical pulses to the pulse transmitter 144. The pulse transmitter 144 is coupled to electrodes 1021 carried by a signal delivery device 1020. In one embodiment, each of these electrodes 1021 is configured to be physically connected to a separate lead, allowing each electrode 1021 to communicate with the pulse generator 143 via a dedicated channel. Accordingly, the pulse generator 143 may have multiple channels, with at least one channel associated with each of the electrodes 1021. Suitable components for the power supply 141, the integrated controller 142, the external controller 146, the pulse generator 143, and the pulse transmitter 144 are known to persons skilled in the art of implantable medical devices.

The pulse system 140 can be programmed and operated to adjust a wide variety of stimulation parameters, for example, which electrodes 1021 are active and inactive, whether electrical stimulation is provided in a unipolar or bipolar manner, and/or how stimulation signals are varied. In particular embodiments, the pulse system 140 can be used to control the polarity, frequency, duty cycle, amplitude, and/or spatial and/or topographical qualities of the stimulation. The stimulation can be varied to match, approximate, or simulate naturally occurring burst patterns (e.g., theta-burst and/or other types of burst stimulation), and/or the stimulation can be varied in a predetermined, pseudorandom, and/or other aperiodic manner at one or more times and/or locations. The signals can be delivered automatically, once initiated by a practitioner. The practitioner (and, optionally, the patient) can override the automated signal delivery to adjust, start, and/or stop signal delivery on demand.

In particular embodiments, the pulse system 140 can receive information from selected sources, with the information being provided to influence the time and/or manner by which the signal delivery parameters are varied. For example, the pulse system 140 can communicate with a database 170 that includes information corresponding to reference or target parameter values. Sensors 160 can be coupled to the patient to provide measured or actual values corresponding to one or more parameters. The sensors 160 can be coupled to the patient's central nervous system (e.g., to the patient's motor cortex) to detect brain activity corresponding to incipient and/or actual hypertonicity behaviors. In particular embodiments, the sensors 160 can include ECoG or EEG sensors. In another embodiment, the sensors 160 can be peripheral sensors that detect muscle tension and/or spastic motion. For example, the sensors can include EMG sensors, accelerometers, or other motion detectors. In any of these embodiments, the measured values of the parameter can be compared with the target value of the same parameter (e.g., an acceptable level of rigidity or spasticity), and the pulse system 140 can be activated if the measured value differs from the target value by more than a threshold amount. Accordingly, this arrangement can be used in a closed-loop fashion to control when stimulation is provided and when stimulation may cease. In one embodiment, some electrodes 1021 may deliver electromagnetic signals to the patient while others are used to sense the activity level of a neural population. In other embodiments, the same electrodes 1021 can alternate between sensing activity levels and delivering electrical signals. In either of these particular embodiments, information received from the signal delivery device 1020 can be used to determine the effectiveness of a given set of signal parameters and, based upon this information, can be used to update the signal delivery parameters and/or halt the delivery of the signals.

In other embodiments, other techniques can be used to provide patient-specific feedback. For example, a magnetic resonance chamber 180 can provide information corresponding to the locations at which a particular type of brain activity is occurring and/or the level of functioning at these locations, and can be used to identify additional locations and/or additional parameters in accordance with which electrical signals can be provided to further increase functionality. Accordingly, the system can include a direction component configured to direct a change in an electromagnetic signal applied to the patient's brain based at least in part on an indication received from one or more sources. These sources can include a detection component (e.g., the signal delivery device and/or the magnetic resonance chamber 880).

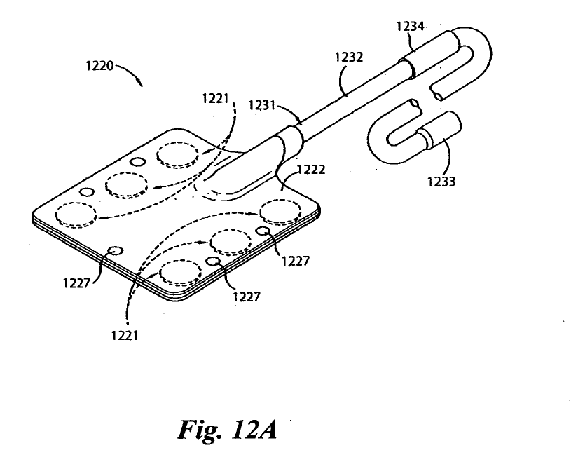

One aspect of the signal delivery device 1020 shown in FIG. 10 is that it can include a support member 1022 that carries multiple electrodes 1021 spaced apart along the generally linear axis. This arrangement can be used to provide electrical signals to multiple target neural populations, and/or to determine a particularly efficacious target neural population by trial and error. FIG. 11 illustrates the signal delivery device 1020 positioned over the left hemisphere 101 of the patient's brain 100, so as to provide some electrodes 1021 over the first target neural population 104a, and others over the second neural target neural population 104b. Accordingly, the same signal delivery device 1020 can apply signals to multiple sites, with power to each of the electrodes 1021 controlled individually so as to provide signals to the appropriate site at the appropriate time and in accordance with the appropriate signal delivery parameters.

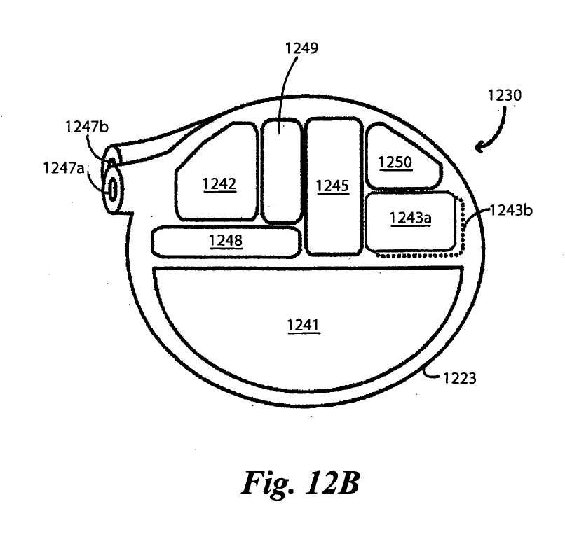

In other embodiments, the system can include signal delivery devices having other configurations. For example, FIG. 12A is a top, partially hidden isometric view of a signal delivery device 1220, configured to carry multiple cortical electrodes 1221 in accordance with another embodiment. The electrodes 1221 can be carried by a flexible support member 1222 to place each electrode 1221 in contact with a target neural population of the patient when the support member 1222 is implanted. Electrical signals can be transmitted to the electrodes 1222 via leads carried in a communication link 1231. The communication link 1231 can include a cable 1232 that is connected to the pulse system 140 (FIG. 10) via a connector 1233, and is protected with a protective sleeve 1234. Coupling apertures or holes 1227 can facilitate temporary attachment of the signal delivery device 1220 to the dura mater at, or at least proximate to, a target neural population. The electrodes 1221 can be biased cathodally and/or anodally. In an embodiment shown in FIG. 12, the signal delivery device 1220 can include six electrodes 1221 arranged in a 2×3 electrode array (i.e., two rows of three electrodes each), and in other embodiments, the signal delivery device 1220 can include more or fewer electrodes 1221 arranged in symmetrical or asymmetrical arrays. The particular arrangement of the electrodes 121 can be selected based on the region of the patient's brain that is to be stimulated, and/or the patient's condition.

FIG. 12B is an internal block diagram of a system 1230 configured in accordance with another embodiment of the invention. The system 1230 can include multiple pulse generators 1243a, 1243b and multiple outputs 1247a, 1247b. Accordingly, the system 1230 may be coupled to two or more signal delivery devices (e.g., two of the devices 1220 shown in FIG. 12A) to apply electromagnetic signals to different target neural populations in one or more manners, which may depend upon the nature or extent of a patient's neurologic dysfunction and/or other embodiment details. The different target neural populations may reside in a variety of anatomical locations, as discussed above. For example, a first and a second target neural population may reside a) in the same or different brain hemispheres; b) in the brain and in the spinal cord; or c) at a central nervous system location and at a peripheral nervous system location. A system having multiple pulse generators 1243a, 1243b may stimulate different neural populations simultaneously or separately, in an independent or correlated manner. One or both pulse generators 1243a, 1243b may generate stimulation signals in various manners described herein.

Other features of the system 1230 include a hermetically sealed housing 1223 that houses a power source 1241 as well as a controller 1242, a telemetry and/or communication unit 1245, and a switching unit 1250. Depending upon embodiment details, the system 1230 may further comprise at least one programmable computer medium (PCM) 1248, which may be coupled to the controller 1242, the telemetry/communication unit 1245, the pulse generators 1243a, 1243b, and/or the switching unit 1250. The system 1230 may additionally comprise at least one timing unit 1249.

The power source 1241 can include a charge storage device such as a battery. In some embodiments, the power source 1241 may additionally or alternatively comprise another type of device for storing charge or energy, such as a capacitor. The controller 1242, the PCM 1249, the telemetry/communication unit 1245, the pulse generators 1243a, 1243b, the switching unit 1250, and/or the timing unit 1249 may include integrated circuits and/or microelectronic devices that synergistically produce and manage the generation, output, and/or delivery of stimulation signals. In certain embodiments, one or more elements within the system 1230 (e.g., the communication unit 1245, the pulse generators 1243a, 1243b, the switching unit 1250, and/or other elements) may be implemented using an Application Specific Integrated Circuit (ASIC).

The timing unit 1249 may include a clock or oscillator and/or circuitry associated therewith configured to generate or provide a set of timing reference signals to the controller 1242, the PCM 1248, the telemetry/communication unit 1245, the pulse generators 1243a, 1243b, the switching unit 1250, and/or one or more portions, subelements, or subcircuits of the system 1230. Such elements, subelements, and/or subcircuits may correlate or synchronize one or more operations to one or more timing reference signals, including the generation of other signals in a manner understood by those skilled in the art.

The controller 1242 may control, manage, and/or direct the operation of elements within the system 1230, e.g., on a continuous, near-continuous, periodic, or intermittent basis depending upon embodiment details. The controller 1242 may include one or more portions of an integrated circuit such as a processing unit or microprocessor, and may be coupled to the programmable computer medium (PCM) 1248. The PCM 1248 may comprise one or more types of memory including volatile and/or nonvolatile memory, and/or one or more data or signal storage elements or devices. The PCM 1248 may store an operating system, program instructions, and/or data. The PCM 1248 may store treatment program information, system configuration information, and stimulation parameter information that specifies or indicates one or more manners of generating and/or delivering stimulation signals in accordance with particular embodiments of the invention.

The switching unit 1250 can include a switch matrix and/or a set of signal routing or switching elements that facilitate the application, delivery, and/or routing of stimulation signals to one or more sets of electrode assemblies, electrical contacts, and/or signal transfer devices at any given time. In one embodiment, the switching unit 1250 may facilitate the electrical activation of particular electrode assemblies, contacts, and/or signal transfer devices, possibly while other such elements remain electrically inactive or electrically float.

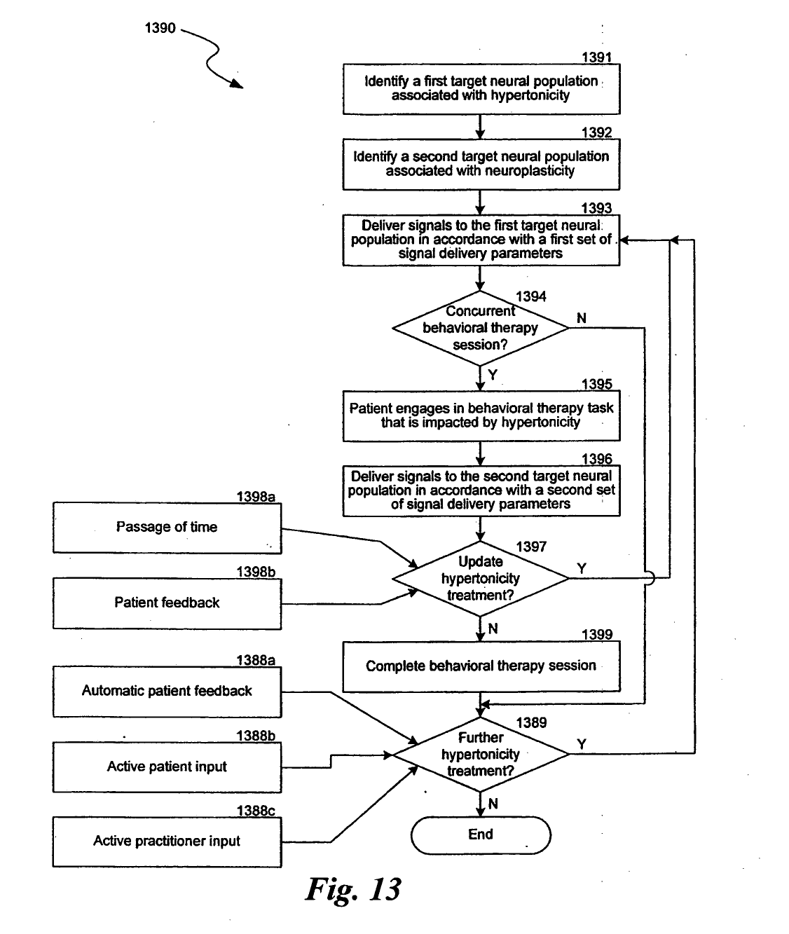

FIG. 13 illustrates a process 1390 for providing treatment to multiple neural populations in accordance with an embodiment of the invention. The process 1390 can include identifying a first target neural population associated with hypertonicity (process portion 1391) and identifying a second target neural population associated with neuroplasticity (process portion 1392). In process portion 1393, signals are delivered to the first target neural population in accordance with the first set of signal delivery parameters. In process portion 1394, it is determined whether the patient is undergoing an adjunctive behavioral therapy session. If not, the process jumps to process portion 1389, where it is determined whether further hypertonicity treatment is to be delivered. This determination can be made based on inputs including automatic patient feedback (process portion 1388a), active patient input (process portion 1388b) and/or active practitioner input (process portion 1388c). Automatic patient feedback can include information that is automatically provided by patient sensors (e.g., peripheral muscle sensors, and/or central nervous system sensors). Active patient input and active practitioner input refer to non-automated (e.g., manual) inputs provided by the patient and the practitioner, respectively. If additional treatment is to be provided, the process returns to process 1393 for additional signal delivery to the first target neural population. If not, the process ends.

If, in process portion 1394, the patient is undergoing a behavioral therapy session, then in process portion 1395, the patient engages in a behavioral therapy task that may be impacted by hypertonicity. Engaging the patient can include having the patient think about, attempt, observe and/or execute a motor task. In process portion 1396, signals are delivered to the second target neural population in accordance with a second set of signal delivery parameters. Signals delivered in accordance with the second set of signal delivery parameters are intended to facilitate the patient's neural plasticity. In process portion 1397, it is determined whether or not to update the hypertonicity treatment. If so, the process 1390 returns to process portion 1393 for additional signal delivery to the first target neural population. The determination as to whether to update the hypertonicity treatment can be made based on the passage of time (process portion 1398a) or other factors, for example, patient feedback (process portion 1398b). The patient feedback can be provided automatically by sensors, such as those described above.

If the patient's hypertonicity treatment is not to be updated in process portion 1397, then the behavioral therapy is completed in process portion 1399, and then in process portion 1389, it is determined whether further hypertonicity treatment is to be delivered to the patient. As described above, this determination can be made based on inputs including automatic patient feedback (process portion 1388a), active patient input (process portion 1388b) and/or active practitioner input of (process portion 1388c). As discussed above, if additional hypertonicity treatment is called for, the process returns to process portion 1393, and if not, the process ends.

One feature of an embodiment of the foregoing process is that the treatment of the patient's hypertonicity can be combined with a treatment that facilitates the patient's functional development or recovery (e.g., in association with neuroplastic processes). An advantage of this arrangement is that, in at least some cases, the patient's hypertonicity may interfere with the patient's recovery from dysfunctions such as stroke, Parkinson's disease, and/or other conditions. By combining these treatments, which may require different treatment parameters (e.g., different signal polarities, frequencies, and/or current or voltage levels), not only can the patient's hypertonicity be addressed, but in doing so, the patient's ability to recover from other dysfunctions may be enhanced.

When multiple electromagnetic treatments are applied to the patient to address hypertonicity as well as one or more other dysfunctions, the practitioner may select different signal application modalities for different treatments, which may involve implanted electrical stimulation, nonimplanted electrical stimulation, or nonimplanted magnetic stimulation. For example, to address patient hypertonicity, the practitioner may select implanted cortical stimulation or transcranial magnetic stimulation (TMS). To address other dysfunctions, the practitioner may select implanted cortical stimulation or transcranial direct current stimulation (tDCS). In other embodiments, the practitioner may select TMS or tDCS to address hypertonicity, and nonimplanted or implanted stimulation devices to address other dysfunctions.

In a particular embodiment, the practitioner can select tDCS to address the effect(s) of paradoxical facilitation (described above with reference to Example 8) upon functional development or recovery. Because it is fairly simple to change the signal polarity of a tDCS device, the practitioner may use the device set at one polarity (e.g. cathodal tDCS for approximately 5-20 minutes) to inhibit portions of the healthy hemisphere (e.g., motor-related cortical regions) from exerting influence upon or taking over functions of the dysfunctional hemisphere. The practitioner may further use tDCS at the opposite polarity (e.g., anodal tDCS for approximately 15-30 minutes) to encourage a neuroplastic response within a portion of the dysfunctional affected hemisphere (e.g., using an electrode positioned above or approximately above the hand knob or other motor cortex region, as described by Hummel and Cohen in “Improvement of Motor Function with Noninvasive Cortical Stimulation in a Patient with Chronic Stroke”, Neurorehabilitation and Neurorepair 19(1), 2005, p. 14-19, incorporated herein by reference in its entirety), and/or use an implanted device in a manner described above to apply electrical signals to portions of the dysfunctional hemisphere. Accordingly, the particular combination selected by the practitioner will likely depend upon factors that include the particular dysfunction(s) to be addressed, the particular patient, and/or other factors.

From the foregoing, it will be appreciated that specific embodiments of the invention have been described herein for purposes of illustration, but that various modifications may be made without deviating from the invention. For example, the signal delivery devices may have configurations other than those described above, and/or the signal delivery parameters may have values other than those described above. Embodiments of additional representative systems and methods are included in the following pending U.S. Applications, all of which are incorporated herein by reference: application Ser. No. 10/260,227, filed Sep. 27, 2002; and application Ser. No. 10/606,202, filed Jun. 24, 2003. Certain aspects of the invention described in the context of particular embodiments maybe combined or eliminated in other embodiments. For example, the patient may receive signals directed to hypertonicity alone in one embodiment, or in combination with signals directed to enhancing neuroplasticity in other embodiments. Further, while advantages associated with certain embodiments of the invention have been described in the context of those embodiments, other embodiments may also exhibit such advantages, and not all embodiments need necessarily exhibit such advantages to fall within the scope of the invention. Accordingly, the invention is not limited except as by the appended claims.

I/We claim:

1. A method for treating a patient, comprising:
identifying a cortical target neural population of the brain associated with hypertonicity in a patient; and
reducing or eliminating patient hypertonicity by applying electromagnetic signals to the target neural population.

2. The method of claim 1 wherein applying electromagnetic signals includes applying electrical signals from a signal delivery device implanted within the patient's skull.

3. The method of claim 1 wherein applying electromagnetic signals includes applying anodal signals.

4. The method of claim 1 wherein applying electromagnetic signals includes applying cathodal signals.

5. The method of claim 1 wherein applying electromagnetic signals includes applying electromagnetic signals to a cortical structure of the brain and triggering signals that propagate to a sub-cortical structure.

6. The method of claim 1 wherein applying electromagnetic signals includes applying excitatory electromagnetic signals to first motor neurons that in turn inhibit second motor neurons, the second motor neurons including alpha motor neurons.

7. The method of claim 1 wherein applying electromagnetic signals includes applying electrical signals to upper neurons at the cortex of the patient's brain or to neurons in synaptic communication with the upper neurons, to have an inhibitory effect on lower alpha motor neurons that are in synaptic communication with the upper motor neurons.

8. The method of claim 1 wherein applying electromagnetic signals includes applying electromagnetic signals to at least one of the motor cortex, the premotor cortex, and the supplementary motor area of the patient's brain.

9. The method of claim 1 wherein applying electromagnetic signals includes applying electromagnetic signals at a subthreshold level.

10. The method of claim 9 wherein applying electromagnetic signals includes applying electromagnetic signals at a level that is from about 70% to about 95% of a movement threshold.

11. The method of claim 1 wherein applying electromagnetic signals includes applying electromagnetic signals at a suprathreshold level.

12. The method of claim 1 wherein applying electromagnetic signals includes applying electromagnetic signals prior to engaging the patient in a behavioral therapy task.

13. The method of claim 1, further comprising engaging the patient in an adjunctive therapy task.

14. The method of claim 13 wherein engaging the patient includes having the patient think about taking a physical action.

15. The method of claim 13 wherein engaging the patient includes having the patient at least attempt a physical action.

16. The method of claim 13 wherein engaging the patient includes having the patient observe an action.

17. The method of claim 1, further comprising engaging the patient in a chemical substance therapy.

18. The method of claim 1 wherein applying electromagnetic signals includes applying electromagnetic signals simultaneously with engaging the patient in a behavioral therapy task.

19. The method of claim 1 wherein the electromagnetic signals are first electromagnetic signals, and wherein the method further comprises reducing or eliminating an additional patient dysfunction by applying second electromagnetic signals.

20. The method of claim 19 wherein the target neural population is a first target neural population, and wherein applying second electromagnetic signals includes applying the second electromagnetic signals to a second target neural population different than the first target neural population.

21. The method of claim 19, further comprising applying the second signals intermittently with the first signals.

22. The method of claim 19 wherein applying the second electromagnetic signals includes facilitating a patient neuroplastic response.

23. The method of claim 19 wherein applying the first signals includes applying the first signals in accordance with a first set of signal delivery parameters and wherein applying the second signals includes applying the second signals in accordance with a second set of signal delivery parameters different than the first set of signal delivery parameters.

24. The method of claim 23 wherein applying the first signals includes applying the first signals at a first polarity and wherein applying the second signals includes applying the second signals at a second polarity opposite the first polarity.

25. The method of claim 1 wherein the electromagnetic signals are first electromagnetic signals provided to a first target neural population to produce an acute reduction in hypertonicity, and wherein the method further comprises:
providing a long-term reduction or elimination of an additional patient dysfunction by applying second electromagnetic signals to a second target neural population different than the first, utilizing a neuroplastic effect; and
providing a long-term reduction or elimination of the patient hypertonicity by applying third electromagnetic signals to the first target neural population, utilizing a neuroplastic effect.

26. The method of claim 25 wherein applying the first signals includes applying the first signals at a level that is from about 70% to about 95% of movement threshold, and wherein applying the second and third signals includes applying the second and third signals at levels of about 25% of movement threshold.

27. The method of claim 1 wherein applying electromagnetic signals includes applying electromagnetic signals for a period of time less than one hour.

28. The method of claim 1 wherein identifying a target neural population includes comparing patient feedback when the patient performs a first task, with patient feedback when the patient performs a second task, the first task evoking hypertonicity, the second task not evoking hypertonicity.

29. The method of claim 1 wherein identifying a target neural population includes comparing patient feedback when the patient performs a first task, with patient feedback when the patient performs a second task, the first task evoking hypertonicity while the patient is affected by a drug, the second task evoking hypertonicity while the patient is not affected by the drug.

30. The method of claim 1 wherein identifying a target neural population includes comparing feedback from the patient when the patient is at rest with feedback from the patient when the patient performs a task that evokes hypertonicity.

31. The method of claim 1 wherein reducing or eliminating hypertonicity includes reducing or eliminating spasticity.

32. The method of claim 1 wherein reducing or eliminating hypertonicity includes reducing or eliminating rigidity.

33. The method of claim 1 wherein applying electromagnetic signals includes applying electromagnetic signals automatically.

34. The method of claim 1 wherein applying electromagnetic signals includes applying electromagnetic signals in response to a practitioner input.

35. The method of claim 1 wherein applying electromagnetic signals includes applying electromagnetic signals in response to a patient input.

36. The method of claim 1 wherein applying electromagnetic signals includes automatically varying a signal delivery parameter over the course of a treatment regimen.

37. The method of claim 1 wherein applying electromagnetic signals includes applying electromagnetic signals on a generally continual basis.

38. The method of claim 1 wherein applying electromagnetic signals includes applying electromagnetic signals automatically in response to a detected indication of actual or incipient hypertonicity.

39. A method for treating a patient, comprising:
identifying a target neural population at a cortical location of a patient's brain, the target neural population being associated with hypertonicity in the patient;
implanting an electrical signal delivery device beneath the patient's skull and proximate to the target neural population; and
reducing or eliminating patient hypertonicity by applying anodal electrical signals to the target neural population to stimulate upper motor neurons that in turn inhibit lower alpha motor neurons with which the upper motor neurons are synaptically coupled.

40. The method of claim 39, further comprising engaging the patient in a behavioral therapy while the patient's hypertonicity is reduced or eliminated under the influence of the anodal electrical signals.

41. A method for treating a patient, comprising:
identifying a target neural population of the central nervous system associated with hypertonicity in a patient;
reducing or eliminating patient hypertonicity by applying first electromagnetic signals to the target neural population in accordance with a first set of signal delivery parameters; and
applying second electromagnetic signals in accordance with a second set of signal delivery parameters different than the first set of signal delivery parameters to an area of the central nervous system to reduce or eliminate an additional patient dysfunction.

42. The method of claim 41 wherein applying the second electromagnetic signals includes applying the second electromagnetic signals to an area of the central nervous system where a change in an intrinsic neural activity is suspected of occurring to carry out a particular physical function and/or cognitive function.

43. The method of claim 41 wherein applying the second electromagnetic signals includes applying the second electromagnetic signals to the target neural population.

44. The method of claim 41 wherein the target neural population is a first target neural population and wherein applying the second electromagnetic signals includes applying the second electromagnetic signals to a second target neural population different than the first target neural population.

45. The method of claim 41 wherein applying the first electromagnetic signals includes applying the first electromagnetic signals with an anodic potential, and wherein applying the second electromagnetic signals includes applying the second electromagnetic signals with a cathodic potential.

46. The method of claim 41, further comprising engaging the patient in an adjunctive behavioral therapy.

47. The method of claim 46 wherein applying the first electromagnetic signals is performed before engaging the patient in the behavioral therapy, and wherein applying the second electromagnetic signals is performed while engaging the patient in the behavioral therapy.

48. The method of claim 46 wherein applying the first electromagnetic signals is performed both before and while engaging the patient in the behavioral therapy, and wherein applying the second electromagnetic signals is performed while engaging the patient in the behavioral therapy.

49. The method of claim 48 wherein applying the first electromagnetic signals while engaging the patient in the behavioral therapy is performed at pre-selected time intervals.

50. The method of claim 48 wherein applying the first electromagnetic signals while engaging the patient in the behavioral therapy is performed in response to an indication that the effect of the first electromagnetic signals applied before the behavioral therapy has diminished.

51. The method of claim 41 wherein the intrinsic neural activity arises in association with a naturally occurring physiological process that facilitates at least partial functional recovery following neurologic damage.

52. A patient treatment system, comprising:
a signal delivery device configured to deliver electromagnetic signals to a cortical brain structure of a patient;
a sensor coupleable to the patient, the sensor being configured to detect an indication of incipient or actual patient hypertonicity; and
a controller coupled to the signal delivery device and the sensor to receive sensor signals corresponding to the indication, and direct the delivery of electromagnetic signals to the patient, based at least in part on the sensor signals.

53. The system of claim 52 wherein the signal delivery device includes at least one electrode carried by a support member and configured to be implanted beneath the patient's skull.

54. The system of claim 52 wherein the sensor includes an accelerometer configured to be coupled to the patient to detect patient movement.

55. The system of claim 52 wherein the sensor includes an EEG sensor.

56. The system of claim 52 wherein the sensor includes an EMG sensor.
